(19) United States
(12) Patent Application Publication
(10) Pub. No.: US 2007/0248684 A1
(43) Pub. Date: Oct. 25, 2007

(54) Pharmaceutical formulations of oxcarbazepine and methods for its preparation

(76) Inventors: Sigal Blau, Raanana (IL); Ilan Zalit, Rosh Ha Ayin (IL); Gershon Kolatkar, Petah Tiqva (IL); Carolina Abrutzky Azaria, Kfar Saba (IL)

Correspondence Address: KENYON & KENYON LLP, ONE BROADWAY, NEW YORK, NY, 10004, US

(21) Appl. No.: 11/701,180

(22) Filed: Jan. 31, 2007

Related U.S. Application Data
(63) Continuation-in-part of application No. 11/350,606, filed on Feb. 8, 2006.
(60) Provisional application No. 60/764,134, filed on Jan. 31, 2006.
(60) Provisional application No. 60/860,333, filed on Nov. 20, 2006.
(60) Provisional application No. 60/897,361, filed on Jan. 24, 2007.

(51) Int. Cl. A61K 9/16

(52) U.S. Cl. 424/499

(57) ABSTRACT

The present invention provides a pharmaceutical composition comprising oxcarbazepine and at least one pharmaceutical excipient, wherein the oxcarbazepine in the composition has a broad particle size distribution. The broad particle size distribution of oxcarbazepine in the pharmaceutical composition is preferably a multi-modal oxcarbazepine particle size distribution, preferably with an enhanced oxcarbazepine dissolution rate.

### CROSS REFERENCE TO RELATED APPLICATIONS

The present application is a Continuation-in-Part of U.S. patent application Ser. No. 11/350,606, filed Feb. 8, 2006. The present application also claims the benefit of the following U.S. Provisional Patent Application Nos. 60/764,134, filed Jan. 31, 2006; 60/860,333, filed Nov. 20, 2006; and 60/(TBA), filed Jan. 24, 2007 (with the title: Pharmaceutical formulation of Oxcarbazepine and methods for its preparation). The contents of these applications are incorporated herein by reference.

### FIELD OF THE INVENTION

The present invention relates to pharmaceutical formulations. More particularly, the present invention concerns formulations comprising oxcarbazepine and methods for preparing these pharmaceutical formulations. Considering the very close similarity between oxcarbazepine and carbamazepine, including their similar low solubility, the present invention is equally applicable to pharmaceutical formulations comprising carbamazepine.

### BACKGROUND OF THE INVENTION

Oxcarbazepine (10-oxo-10,11-dihydro-5H-dibenz[b,f]azepine-5-carboxamide) of the general formula: 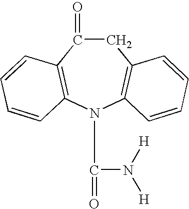
has valuable therapeutic benefits and acts as a central nervous system depressant. Currently it is being marketed as TRILEPTAL®, for the treatment of epilepsy. According to the prescribing information for TRILEPTAL®, the pharmacological benefit of oxcarbazepine is primarily exerted through the 10-hydroxy metabolite of oxcarbazepine. In vitro studies indicate that the metabolite blocks voltage sensitive sodium channels, which results in the stabilization of hyperexcited neural membranes, inhibition of repetitive neuronal firing, and diminution of propagation of synaptic impulses. These actions are thought to be important in the prevention of seizure spread in the brain. U.S. Pat. No. 5,658,900, incorporated herein by reference, further describes the use of oxcarbazepine to treat Parkinson's disease.

Oxcarbazepine, an antiepileptic drug, is a white to yellowish crystalline powder that is practically insoluble in water. There is a need to enhance the dissolution rate and the bioavailability of oxcarbazepine formulations. It has long been known in the art that the dissolution and bioavailability of poorly soluble drugs may be enhanced by using small particles of such drugs, and applying a narrow particle size distribution which is considered normal formulation practice. In line herewith, Schlutermann (US patent application 2003/0190361) describes a formulation comprising oxcarbazepine of fine particle size and a narrow size distribution. The particles are characterized as having a median particle size of approximately 2 to 12 microns and leave a maximum residue on a 40 micron sieve of up to 5%. Schlutermann also describes a film-coated tablet comprising oxcarbazepine particles with the same characteristics. Further, EP0646374 describes a color stable formulation of oxcarbazepine, which is coated with two layers, wherein each layer contains white pigments.

A common method of grinding drugs employs a jet mill. The jet mill uses compressed air to produce micronized particles from large dry particles. The mill is designed in such a way that the powdered particles exit the milling chamber and are collected in a collection vessel. Waste of fine particles is also produced during the process and is collected in a filter bag. Grinding processes such as this for example are costly in both their investment, as grinding is a time consuming process, and in operating costs. In addition, such processes result in substantial losses of the drug during grinding. Furthermore, safety considerations should be taken into account during the air jet milling, due to the active pharmaceutical ingredient (API) dust formation accompanying the process. These concerns become more problematic for pharmaceutical compositions comprising oxcarbazepine due to the relatively large recommended daily dose of 1200 mg/day.

Further, Sehgal et al. (WO/2002/094774) describes a method to enhance the dissolution rate of oxcarbazepine by adding a wetting agent into the formulation. The addition of such wetting agent to the oxcarbazepine formulation, enhances the dissolution rate in-vitro.

In preferred embodiments, the present invention provides a process of preparing an oxcarbazepine formulation with a sufficiently high dissolution rate, suitable bioavailability, and bioequivalent to the marketed as TRILEPTAL®.

### SUMMARY OF THE INVENTION

The present invention provides a pharmaceutical composition comprising, a) oxcarbazepine, and b) at least one pharmaceutical excipient, wherein the oxcarbazepine in the composition has a broad particle size distribution. The broad particle size distribution may comprise a multi-modal oxcarbazepine particle size distribution.

Further, there is provided a method for preparing a pharmaceutical composition of the invention comprising the steps of:

a) providing oxcarbazepine having a broad particle size distribution;

b) providing at least one excipient;

c) combining the oxcarbazepine with the at least one excipient.

Also, there is provided a method of preparing a granular composition comprising oxcarbazepine having a broad particle size distribution which comprises the following steps of

a) providing oxcarbazepine with broad particle size distribution, optionally comprising two or more populations of oxcarbazepine having different particle size distributions;

b) providing at least one excipient;

c) forming at least one granulate comprising at least one of said oxcarbazepine populations; and

d) mixing the granulated oxcarbazepine with any remaining ungranulated oxcarbazepine and one or more excipients to form a final granulate blend. When preparing tablets from the granular composition the method further comprises the steps of

e) optionally mixing the final granulate blend with one or more excipients to form a tabletting mixture;

f) pressing either the tabletting mixture or the final granulate blend into tablets; and optionally

g) coating the tablets.

The present invention also provides a pharmaceutical composition comprising, a) spray granulated oxcarbazepine, and b) at least one pharmaceutical excipient.

In another aspect the present invention provides a pharmaceutical composition comprising, a) oxcarbazepine with broad particle size distribution, and b) at least one pharmaceutical excipient. Optionally, the oxcarbazepine in the composition has at least two populations of different particle sizes and at least one of the populations of oxcarbazepine particles comprises spray granulated oxcarbazepine.

Further, there is provided a pharmaceutcial composition comprising a) oxcarbazepine having a broad particle size distribution; b) a hydrophilic polymer, preferably Hydroxypropylmethylcellulose (hypromellose, HPMC); and c) at least one pharmaceutical excipient, wherein the weight ratio of hydrophilic polymer to oxcarbazepine in the composition is from about 1:3 to about 1:25. Preferably, the broad particle size distribution of oxcarbazepine is a multi-modal particle size distribution.

In another aspect there is provided a pharmaceutical composition comprising a) oxcarbazepine having a broad particle size distribution; and b) at least one pharmaceutical excipient, wherein the pharmaceutical composition is bioequivalent to Trileptal®. Preferably, the broad particle size distribution of oxcarbazepine is a multi-modal particle size distribution.

In another aspect there is provided a pharmaceutical composition comprising a) oxcarbazepine having a broad particle size distribution; and b) at least one pharmaceutical excipient, wherein the disintegration time of the pharmaceutical composition is less than about 30 minutes. Preferably, the broad particle size distribution of oxcarbazepine is a multi-modal particle size distribution.

Further, there is provided a method of preparing a granular composition comprising at least two populations of different oxcarbazepine particle sizes wherein at least one population of oxcarbazepine particles is spray granulated comprising the following steps of

a) providing oxcarbazepine which comprises two or more populations of oxcarbazepine having different particle size distributions;

b) mixing at least one population of oxcarbazepine particles with one or more excipients and forming at least one spray granulate; and

c) mixing the at least one spray granulated oxcarbazepine population of particle sizes with any remaining non-spray granulated population of oxcarbazepine particle sizes, which together form an oxcarbazepine mixture of the total amount of oxcarbazepine in the composition.

When preparing tablets from the granular composition the method further comprises at least one of the following steps d) and e);

d) mixing the oxcarbazepine mixture with one or more excipients and forming a final granulate;

e) mixing the final granulate with one or more excipients to form a tabletting mixture; and then,

f) pressing the tabletting mixture into tablets; and optionally

g) coating the tablets.

Further, there is provided a method of preparing a granular composition comprising at least two populations of different oxcarbazepine particle sizes wherein at least one population of oxcarbazepine particles is spray granulated comprising the following steps of

a) providing oxcarbazepine which comprises two or more populations of oxcarbazepine having different particle size distributions; and

b) spraying at least one population of oxcarbazepine particles on a mixture of one or more excipients and any remaining population of oxcarbazepine and forming at least one spray granulate. When preparing tablets from the granular composition the method further comprises the following steps

c) optionally mixing the final granulate with one or more excipients to form a tabletting mixture;

d) pressing the tabletting mixture into tablets; and optionally

e) coating the tablets.

Further, there is provided a method of preparing a granular composition comprising at least two populations of different oxcarbazepine particle sizes wherein at least two populations of oxcarbazepine particles are spray granulated comprising the following steps of

a) providing oxcarbazepine which comprises two or more populations of oxcarbazepine having different particle size distributions;

b) spraying separately at least two populations of oxcarbazepine particles with one or more excipients and forming at least two spray granulates; and

c) mixing the at least two spray granulated oxcarbazepine populations of oxcarbazepine particle sizes. When preparing tablets from the granular composition the method further comprises at least one of the following steps

d) optionally mixing the oxcarbazepine mixture with one or more excipients and forming a final granulate;

e) mixing the final granulate with one or more excipients to form a tabletting mixture;

f) pressing the tabletting mixture into tablets; and optionally

g) coating the tablets.

Further, there is provided a method of preparing a granular composition comprising oxcarbazepine with broad particle size distribution which comprises the following steps of

a) providing oxcarbazepine which comprises broad particle size distributions; and

b) spraying the oxcarbazepine particles with one or more excipients and forming a spray granulate. When preparing tablets from the granular composition the method further comprises at least one of the following steps

c) optionally mixing the granulate with one or more excipients to form a tabletting mixture;

d) pressing the tabletting mixture into tablets; and optionally

e) coating the tablets.

In another aspect the present invention also provides a pharmaceutical composition comprising oxcarbazepine with a broad particle size distribution and having a dissolution profile such that

a) no more than about 30% of the total amount of oxcarbazepine is dissolved from the composition after 35 minutes of measurement in a dissolution apparatus;

b) from about 30% to about 50%, preferably about 40% or more, of the total amount of oxcarbazepine is dissolved from the composition after 50 minutes of measurement in a dissolution apparatus; and

c) from about 30% to about 50% of the total amount of oxcarbazepine is dissolved from the composition after 60 minutes of measurement in a dissolution apparatus system that simulates the gastrointestinal environment.

In another aspect, the present invention provides a method of treating a patient suffering from epileptic seizures or Parkinson's disease or neuropathic pain comprising administering a therapeutically effective amount of oxcarbazepine in a pharmaceutical composition comprising a multi-modal oxcarbazapine particle size distribution.

In another embodiment of the present invention there is also provided a pharmaceutical composition, a method of preparing the pharmaceutical composition and method of treatment as in any of the above embodiments wherein carbamazepine is substituted for the oxcarbazepine.

### BRIEF DESCRIPTION OF THE DRAWINGS

FIG. 1: Dissolution profile of oxcarbazepine tablets (prepared according to examples 1 and 2) in comparison to a TRILEPTAL® bioequivalent formulation under simulated gastrointestinal conditions.

FIG. 2: Dissolution profile of oxcarbazepine tablets (prepared according to examples 3 and 4) in comparison to a TRILEPTAL® bioequivalent formulation and a non-bioequivalent formulation (prepared according to example 7) under simulated gastrointestinal conditions.

FIG. 3: Dissolution profile of oxcarbazepine tablets (prepared according to examples 5 and 6) in comparison to a TRILEPTAL® bioequivalent formulation and a non-bioequivalent formulation (prepared according to example 7) under simulated gastrointestinal conditions.

FIG. 4: Dissolution profile of oxcarbazepine tablets (prepared according to examples 8 and 9) in comparison to a TRILEPTAL® bioequivalent formulation under simulated gastrointestinal conditions.

FIG. 5: Dissolution profile of oxcarbazepine tablets (prepared according to examples 10 and 11) in comparison to a TRILEPTAL® bioequivalent formulation and a non-bioequivalent formulation (prepared according to example 7) under simulated gastrointestinal conditions.

FIG. 6: Dissolution profile of oxcarbazepine tablets (prepared according to examples 12 and 13) in comparison to a TRILEPTAL® bioequivalent formulation and a non-bioequivalent formulation (prepared according to example 7) under simulated gastrointestinal conditions.

FIG. 7: Dissolution profile of oxcarbazepine tablets (prepare according to examples 15 and 16) in comparison to a TRILEPTAL® bioequivalent formulation and a non-bioequivalent formulation.

### DETAILED DESCRIPTION OF THE INVENTION

As used herein, “broad particle size distribution” means a particular particle size distribution wherein the difference in the value of d(0.5) and d(0.95) is greater than 38 microns, and the d(0.5) value is 35 microns or less. The d-values may be determined by methods such as laser diffraction, sieving or other methods known in the art. For example, the value of d(0.95) may be estimated from the sieve size through which no more than 95% of the particles would pass. As used herein, “d(0.5)” (which may also be written as “d(v, 0.5)” or volume median diameter) represents the particle size (diameter) for which the cumulative volume of all particles smaller than the d(0.5) value in a population is equal to 50% of the total volume of all particles within that population.

In one embodiment, the broad particle size distribution of oxcarbazepine may be characterized by having d(0.5) values ranging from 12 to 35 μm, preferably 13 to 30μ and more preferably from 14 to 25μ. In another embodiment, d(0.5) ranges from 0.01μ to 2.0μ (more preferably from 0.2 to 1.9μ and most preferably from 0.4 to 1.5μ). In another embodiment, d(0.5) ranges from 2-12μ.

Notwithstanding the d(0.5) value, the d(0.95) is preferably greater than 40μ. For example, sieving of the population using a 40μ sieve results with residue of more than 5% on sieve.

The term “multi-modal particle size distribution” and “multi-modal oxcarbazepine particle size distribution” are meant to be understood as either, oxcarbazepine having a particular particle size distribution which is characterized in that a graphic plot of oxcarbazepine particle sizes by volume or weight, displays two or more peak particle sizes. For example a multi-modal particle size distribution of oxcarbazepine may be prepared by mixing at least two populations of oxcarbazepine characterized by different particle size distributions. Preferably, the multi-modal particle size distribution of the present invention is a bi-modal particle size distribution. In a preferred bi-modal particle size distribution the difference in the two modes of populations of oxcarbazepine particles is at least about 12 microns, preferably at least about 13 microns. A particle size distribution as described in the present invention can be determined by various conventional methods of analysis, such as Laser light scattering, laser diffraction, sedimentation methods, pulse methods, electrical zone sensing, sieve analysis and optical microscopy (usually combined with image analysis). The evaluation of particle size distribution of the multi modal particle size distribution, prepared by a mix of at least two population of oxcarbazepine, can be performed by preparing a mix of multi oxcarbazepine particle size populations at appropriate ratio and analyze their particle size distribution by laser diffraction method. Another option to evaluate the multi modal particle size distribution (PSD) is by mathematical calculation of d(0.1), d(0.5) and d(0.9) which is performed by calculation of the PSD weighted mean (average) of each of the oxcarbazepine populations. See, e.g., Table 1.

As used herein the term “large drug particles” refers to populations having a d(0.5) of at least about 12 microns, preferably at least about 13 microns. Preferably, a population of large oxcarbazepine particles is characterized as a population selected from the group consisting of, a population of oxcarbazepine particles with a d(0.5) of at least 13 microns, a population of unground particles, particles that do not pass a 40 micron sieve, and mixtures thereof. Preferably, a population of large oxcarbazepine particles is characterized as a population having a d(0.5) oxcarbazepine particle size of at least about 13 microns, and the population of large oxcarbazepine particles comprises more than about 5% of particles that do not pass a 40 micron sieve. Thus, sieving a population of large oxcarbazepine particles' using a 40 micron sieve results in a residue of more than 5% on sieve or d(0.95) is greater than 40 microns. Preferably, this amount of residue on a 40 micron sieve is more than about 25%, more preferably more than about 50%, for such population of large oxcarbazepine particles. As such the d(0.5) of oxcarbazepine of a population of large oxcarbazepine particles may preferably be at least 30 microns, more preferably at least 45 microns, even more preferably at least 55 microns. A population of large oxcarbazepine particles may also be characterized by having d(0.5) values range from about 12 to 120μ, preferably from about 60 to 90μ. The term “unground particles” refers to particles obtained from the synthesis of oxcarbazepine (the active pharmaceutical ingredient, API) that have not been exposed to grinding or have been exposed to only minimal grinding. Unground oxcarbazepine particles may have a d(0.5) of more than 13 microns. In one example, unground particles may be characterized by d(0.1), d(0.5) and d(0.9) of about 21, 71 and 248 microns.

In contrast, as used herein the term “small drug particles” generally means populations of drug particles with d(0.5) values smaller than 6 microns, preferably smaller than 3 microns, more preferably smaller than 2 microns. Preferably, sieving of the same population using a 40 μm sieve results in a residue of less than 5% on the sieve or d(0.95) is less than 40 microns.

Also as used herein the term “spray-granulated oxcarbazepine” refers to granulated populations of oxcarbazepine particles that have been granulated by spraying an oxcarbazepine dispersion on a powder carrier.

An important aspect in formulating an oxcarbazepine dosage form for use in medical treatments is achieving an adequate dissolution rate of the poorly soluble active pharmaceutical ingredient oxcarbazepine from a pharmaceutical composition. According to the present invention a dissolution rate similar to that of the TRILEPTAL® bioequivalent formulation (the Trileptal® bioequivalent composition A) is achievable with formulations having oxcarbazepine comprising a broad particle size distribution. Preferred embodiments of the present invention encompass pharmaceutical compositions comprising oxcarbazepine, preferably having a desired dissolution rate and bioavailability using a safer method with less dust formation during the grinding process and/or reduced grinding cost and loss of active pharmaceutical ingredient. One embodiment of the present invention provides a pharmaceutical composition comprising, a) oxcarbazepine, and b) at least one pharmaceutical excipient, wherein the oxcarbazepine in the composition has a broad particle size distribution. Optionally, the broad particle size distribution is a multi-modal oxcarbazepine particle size distribution.

The oxcarbazepine pharmaceutical composition of the present invention is characterized by its broad particle size distribution. Particle size distribution can be determined by various conventional methods of analysis, such as Laser light scattering, laser diffraction, sedimentation methods, pulse methods, electrical zone sensing, sieve analysis and optical microscopy (usually combined with image analysis).

Preferably, the multi-modal oxcarbazepine particle size distribution in the pharmaceutical composition contains at least two populations of particles. Each of these populations differ in their median particle size (or volume median diameter) and have a distinct particle size distribution.

In a preferred embodiment of the invention, the pharmaceutical composition comprises two or more populations of oxcarbazepine particles wherein at least one of these populations is a population of large oxcarbazepine particles. A population of large oxcarbazepine particles is characterized as a population selected from the group consisting of, a population of oxcarbazepine particles with a d(0.5) of at least 13 microns, a population of unground particles, particles that do not pass a 40 micron sieve, and mixtures thereof.

An embodiment of the invention includes a pharmaceutical compositions of the present invention comprising large oxcarbazepine particles in an amount between about 6% by weight, and about 49% by weight of the total amount of oxcarbazepine in the pharmaceutical composition. Further, in this embodiment the pharmaceutical compositions of the present invention comprises at least one population of small oxcarbazepine particles, wherein the oxcarbazepine d(0.5) particle size is smaller than about 6 microns, preferably smaller than about 3 microns, most preferably smaller than 2 micron. Such pharmaceutical composition preferably comprises small oxcarbazepine particles in an amount from about 94% by weight, to about 60% by weight of the total amount of oxcarbazepine in the pharmaceutical composition. Preferably the content of the small oxcarbazepine particles in the multi-modal pharmaceutical composition is adjusted to the content of the large oxcarbazepine particles. Thus, according to one preferred embodiment, the pharmaceutical composition comprises large oxcarbazepine particles in an amount from about 6% to about 40% by weight and small oxcarbazepine particles in an amount from about 94% to about 60% by weight of the total amount of oxcarbazepine in the pharmaceutical composition. More preferably, the pharmaceutical composition comprises large oxcarbazepine particles in an amount from about 10% to about 35% by weight, preferably in an amount selected from about 10%, about 20% and about 35% by weight, and small oxcarbazepine particles in an amount from about 90% to about 65% by weight, preferably in an amount selected from about 90%, about 80%, and about 65% by weight, of the total amount of oxcarbazepine large particles.

Another embodiment of a pharmaceutical composition of the present invention comprises large oxcarbazepine particles in an amount of between about 95% by weight and about 51% by weight of the total amount of oxcarbazepine in the pharmaceutical composition. Further, the pharmaceutical compositions of this embodiment of the present invention comprises at least one population of small oxcarbazepine particles, wherein the d(0.5) is smaller than about 6 microns, preferably smaller than about 3 microns, most preferably smaller than 2 micron. Furthermore, such pharmaceutical compositions comprises small oxcarbazepine particles in an amount from about 5% to about 49% by weight. Preferably the content of the small oxcarbazepine particles in the pharmaceutical composition is adjusted to the content of the large oxcarbazepine particles. Thus, according to one preferred embodiment, the pharmaceutical composition comprises large oxcarbazepine particles in an amount from about 90% to about 60% by weight and small oxcarbazepine particles in an amount from about 10% to about 40% by weight of the total amount of oxcarbazepine in the pharmaceutical composition. More preferably, the pharmaceutical composition comprises large oxcarbazepine particle in an amount from about 85% to about 65% by weight and small oxcarbazepine particles in an amount from about 15% to about 35% by weight of the total amount of oxcarbazepine large particles.

In another embodiment of the present invention, the population of oxcarbazepine particles has a d(0.5) of not more than 2 microns and has a minimum residue on a 40 micron sieve of more than 5%. Alternatively, in another embodiment of the present invention, the population of oxcarbazepine particles has a d(0.5) of 2 to 12 microns and has a minimum residue on a 40 micron sieve of more than 5%. Yet, in another embodiment of the present invention, the oxcarbazepine has a d(0.5) of not less than 13 microns and has a minimum residue on a 40 micron sieve of more than 5%.

Another embodiment of the present invention provides a pharmaceutical composition comprising, a) oxcarbazepine, and b) at least one pharmaceutical excipient, wherein the oxcarbazepine in the composition has a particle size distribution characterized by oxcarbazepine with d(0.5) values ranging from 13 to 40 microns and more than 5% residue on 40 micron sieve. The oxcarbazepine can be from a single population of particles.

Further, in another embodiment of the present invention a pharmaceutical composition comprises spray granulated oxcarbazepine. Preferably, in this embodiment of the present invention such pharmaceutical composition comprises a) oxcarbazepine; and b) at least one pharmaceutical excipient, wherein the oxcarbazepine has a broad particle size distribution. The broad particle size distribution of the oxcarbazepine particles may be a multi-modal particle size distribution. As described such multi-modal particle size distribution comprises two or more populations of oxcarbazepine particles having a distinct particle size distribution. In a pharmaceutical composition of the present invention comprising spray granulated oxcarbazepine one or more of the populations of oxcarbazepine particles having distinct particle size distributions is spray granulated.

In another embodiment of the present invention there is provided a pharmaceutcial composition comprising a) oxcarbazepine having a broad particle size distribution; b) a hydrophilic polymer; and c) at least one pharmaceutical excipient, wherein the weight ratio of the hydrophilic polymer to oxcarbazepine in the composition is from about 1:3 to about 1:25. The hydrophilic polymer is preferably Hydroxypropylmethylcellulose (hypromellose, HPMC). The bioavaliability of an oral tablet may be influenced by disintegration and dissolution. In order to reduce disintegration time of oxcarbazepine formulations a defined amount of hydrophilic polymer was used when small oxcarbazepine is sprayed onto large oxcarbazepine particles in a spray granulation process. A preferred ratio of hydrophilic polymer to oxcarbazepine is from about 1:5 to about 1:22, more preferably from about 1:9 to about 1:20. The total amount of hydrophilic polymer in the pharmaceutical composition of the present invention can also be expressed as not more than about 120 mg, preferably from about 20 mg to about 120 mg, more preferably from about 20 mg to about 50 mg, most preferably from about 30 mg to about 45 mg. Preferably, the total amount of hydrophilic polymer in the pharmaceutical composition of the present invention is not more than about 15% by weight of the composition, more preferably from about 1% to about 15% by weight of the composition, even more preferably about 2% to about 15% by weight of the composition, yet even more preferably from about 2% to about 12% by weight of the composition, even more preferably from about 2.5% to about 10% by weight of the composisition, most preferably from about 2.5% to about 5% by weight. In a most preferred embodiment the total amount of hydrophilic polymer in the pharmaceutical composition is from about 2% to about 4% by weight of the composition. Preferably, the broad particle size distribution of oxcarbazepine is a multi-modal particle size distribution. Preferably, the oxcarbazepine of this embodiment of the present invention comprises at least one population of small particles. This population of small oxcarbazepine particles may be spray granulated when preparing a pharmaceutical composition of the present invention. In a preferred embodiment at least one population of small oxcarbazepine particles is spray granulated. The ratio of the amount of hydrophilic polymer to the amount of small oxcarbazepine particles in such spray granulate is from about 1:3 to about 1:20, preferably from about 1:5 to about 1:18, more preferably from about 1:9 to about 1:17.

The oxcarbazepine pharmaceutical compositions comprising a broad particle size distribution as in the present invention further may contain excipients such as tablet and capsule fillers and diluents (such as microcrystalline cellulose, lactose, starch and tri-basic calcium phosphate), disintegrants (such as starch, croscarmellose sodium and sodium starch glycolate), binders (such as starch, hydroxypropyl methyl cellulose and Povidone), glidant (such as colloidal silicon dioxide), lubricants (such as magnesium stearate, magnesium lauryl sulfate and sodium stearyl fumarate) and surfactants and wetting agents (such as sodium lauryl sulfate, polysorbate and poloxamer).

More particularly, suitable diluents and fillers for use in the pharmaceutical composition of the present invention include microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, potassium chloride, powdered cellulose, sodium chloride, sorbitol and talc.

Further, suitable surfactants for use in the pharmaceutical composition of the invention include poloxamers, polyethylene glycols, polysorbates, sodium lauryl sulfate, polyethoxylated castor oil, and hydroxylated castor oil.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydroxyethyl cellulose, hydroxypropyl cellulose (e.g. Klucel®), hydroxypropyl methyl cellulose (e.g. Methocel®), liquid glucose, maltodextrin, methylcellulose, polymethacrylates, povidone (e.g. Povidone PVP K-30, Kollidon®, Plasdone®), pregelatinized starch, sodium alginate and starch.

A compacted solid pharmaceutical composition may also include the addition of a disintegrant to the composition. Disintegrants include croscarmellose sodium (e.g. Ac Di Sol®, Primellose®), crospovidone (e.g. Kollidon®, Polyplasdone®), microcrystalline cellulose, polacrilin potassium, powdered cellulose, pregelatinized starch, sodium starch glycolate (e.g. Explotab®, Primoljel®) and starch.

Glidants can be added to improve the flowability of a non compacted solid composition and to improve the accuracy of dosing. Excipients that may function as glidants include colloidal silicon dioxide, magnesium trisilicate, powdered cellulose, and talc.

A lubricant can be added to the composition to reduce adhesion and/or ease the release of the product from e.g. the dye. Lubricants include magnesium stearate, calcium stearate, glyceryl monostearate, glyceryl palmitostearate, hydrogenated castor oil, hydrogenated vegetable oil, mineral oil, polyethylene glycol, sodium lauryl sulfate, sodium stearyl fumarate, stearic acid, talc and zinc stearate.

Other excipients that may be incorporated into the formulation include preservatives, antioxidants, or any other excipient commonly used in the pharmaceutical industry.

The solid compositions of the present invention include powders, granulates, aggregates and compacted compositions. The dosages include dosages suitable for oral, buccal, and rectal administration. Although the most suitable administration in any given case will depend on the nature and severity of the condition being treated, the most preferred route of the present invention is oral. The dosages may be conveniently presented in unit dosage form and prepared by any of the methods well known in the pharmaceutical arts.

The present invention also discloses a method for the production of oxcarbazepine pharmaceutical composition with broad particle size distribution. e.g. one of the methods comprises a first step of mixing large oxcarbazepine particles with small oxcarbazepine particles. This mixing can be carried out either prior to granulation or during granulation. Alternatively, at least part of the large size oxcarbazepine particles, for example at least one population of large oxcarbazepine particles, and at least part of the small size oxcarbazepine particles, for example at least one population of small oxcarbazepine particles, are granulated separately and the formed granules are mixed. The mixture can then be combined with additional excipients and pressed into tablets.

According to a preferred embodiment, the process involves milling and optionally de-agglomerating a portion of the total amount of oxcarbazepine to form small drug particles. Preferably, the remaining amount of oxcarbazpine is not milled and used as such forming a population of large drug particles. Alternatively, this remaining amount of oxcarbazepine is only slightly milled forming large drug particles of the multi-modal pharmaceutical composition of the present invention. This slightly milled portion of oxcarbazepine retains a large particle size distribution. Various known methods can be used for milling and de-agglomeration of oxcarbazepine particles. A jet mill, impact mill, ball mill, vibration mill, mortar mill or pin mill may for example be used for milling unground oxcarbazepine. Preferably, the oxcarbazepine is milled in a liquid dispersion by a homogenizer, such as rotor-stator or high pressure homogenizer such as a microfluidizer®, which produces milled oxcarbazepine at a high yield. Preferably, such reduction of the particle size of oxcarbazepine by wet milling is carried out in the presence of a hydrophilic polymer or stabilizer. A preferred hydrophilic polymer or stabilizer is a hypromellose (Hydroxypropylmethylcellulose, HPMC), for example Pharmacoat®.

Preferably, the method of the present invention produces compressed solid dosage forms. There are three well known processes for manufacturing such dosage forms; (i) direct compression, (ii) dry granulation and (iii) wet granulation. There are two well known processes for wet granulation. A wet granulate can be prepared using a mixer and subsequently the wet granulate is dried in order to obtain a dry homogenous granulate. In another method a wet granulate is prepared by spray granulation. In a fluid-bed, spray granulation process, particles and granulate are built up in a fluid bed by spraying a liquid onto fluidized particles. Thus in such process materials are fluidized in the fluid bed dryer and subsequently a solution is sprayed through a nozzle.

The pharmaceutical composition of the present invention may be prepared in any dosage form such as a compressed granulate in the form of a tablet for example. Also, uncompressed granulates and powder mixes that are obtained by the method of the present invention in the pre-compression steps can be simply provided in dosage form of a capsule or sachet. Therefore, dosage forms of pharmaceutical composition of the present invention include solid dosage forms like tablets, powders, capsules, sachets, troches and losenges. The dosage form of the present invention may also be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

Preferably, the production of the pharmaceutical composition comprising a broad particle size distribution of oxcarbazepine comprises spray granulation of at least one population of oxcarbazepine particles, either from small drug particles or from large drug particles. In another preferred embodiment of the preparation method, the process comprises spray granulation of both a population of small and large drug particles together. In this later embodiment, a suitable dissolution rate and bioavailability obtained at reduced grinding cost, higher safety and less loss of active pharmaceutical ingredient are achieved by using pharmaceutical compositions comprising spray-granulated oxcarbazepine. Spray-granulated particles are described in various publications, e.g. on the website of Glatt, a manufacturer of spray granulation equipment. Preferably, spray-granulated oxcarbazepine is a product of spraying a dispersion of oxcarbazepine particles on air-fluidized excipient particles.

In one embodiment, the sprayed oxcarbazepine dispersion comprises relatively large oxcarbazepine particles. These relatively large particles comprise oxcarbazepine particles as may be obtained from synthesis without being milled, for example particles characterized with d(0.1), d(0.5) and d(0.9) of about 21, 71 and 248 microns, respectively, or those which have been subjected to only minimal grinding. Relatively large particles for use in spray granulation may also be described as particles with a d(0.5) size greater than 13 microns, or oxcarbazepine particles that do not pass a 40 micron sieve. Optionally, the pharmaceutical composition of spray granulated oxcarbazepine also comprises oxcarbazepine particles of smaller size which are spray granulated. Optionally, the oxcarbazepine dispersion for use in spray granulation has a multi-modal oxcarbazepine particle size distribution similar to the oxcarbazepine in a multi-modal oxcarbazepine pharmaceutical composition.

In another embodiment, the sprayed oxcarbazepine dispersion comprises relatively small oxcarbazepine particles. These relatively small particles comprise oxcarbazepine particles as are obtained, for example, following a grinding process. Alternatively, such dispersion of relatively small oxcarbazepine particles is obtained by providing a dispersion comprising relatively large oxcarbazepine particles and reducing the particle size of the oxcarbazepine particles in the dispersion, preferably by a high pressure homogenization process. Relatively small particles for use in spray granulation may also be described as particles with a d(0.5) size smaller than 6 microns, preferebly smaller than 3 microns. Optionally, the pharmaceutical composition of spray granulated oxcarbazepine also comprises oxcarbazepine particles of larger size which are spray granulated. Optionally, the oxcarbazepine dispersion for use in spray granulation has a multi-modal oxcarbazepine particle size distribution similar to the oxcarbazepine in a multi-modal oxcarbazepine pharmaceutical composition.

The pharmaceutical compositions of spray-granulated oxcarbazepine comprise at least one excipient selected from the group such as tablet and capsule fillers and diluents (such as microcrystalline cellulose, lactose, starch and tri-basic calcium phosphate), disintegrants (such as starch, croscarmellose sodium and sodium starch glycolate), binders (such as starch, hydroxypropyl methyl cellulose and Povidone), glidant (such as colloidal silicon dioxide), lubricants (such as magnesium stearate, magnesium lauryl sulfate and sodium stearyl fumarate) and surfactants and wetting agents (such as sodium lauryl sulfate, polysorbate and poloxamer). In a preferred embodiment, the sprayed dispersion of oxcarbazepine comprises a binder, such as hypromellose (Hydroxypropylmethylcellulose, HPMC), for example Pharmacoat®.

The present invention also discloses a method for the production of a pharmaceutical composition of spray-granulated oxcarbazepine. The method involves spraying of at least one excipient with a dispersion of oxcarbazepine. First a dispersion of oxcarbazepine is prepared and this dispersion of oxcarbazepine is sprayed onto fluidized excipients particles, which are then dried, preferably rapidly in the fluidizing/suspending gas (typically, air). The spraying process is preferably done by Fluidized bed technology equipment, where the particles are suspended in a vertical column with a rising air stream. While the particles are fluidized, the coating dispersion of oxcarbazepine is sprayed into the column. This spraying can be carried out by any one of three methods; top spray, bottom spray and a “tangential” or powder spray. Preferably the spraying of the oxcarbazepine dispersion is carried out by a top spray method.

Moreover, the preferred spray-granulation method of the present invention differs somewhat from the commonly used spray-granulation method. In the commonly used procedure a granulation solution is sprayed onto a mixture of active and inactive materials which are suspended or fluidized in air. In the preferred method of spray-granulation of the present invention, a dispersion of the active material is sprayed on excipients suspended in air or on a mixture of active and inactive materials.

In particular, when in the above spray granulation method two separate granulations are prepared, one comprising small oxcarbazepine particles and the other comprising large oxcarbazepine particles, a further advantage of the invention is achieved. It is often important to adjust and fine tune the dissolution characteristics of the final pharmaceutical compositions during development. Once the dissolution rate is determined for each granulation, such fine tuning of the dissolution rate for the final pharmaceutical composition becomes extremely simple. A mere mixing of the two or more granulates in appropriate proportions will achieve the desired resulting dissolution rate. When a higher rate of dissolution is required, the mixture of granulates will comprise a proportionately higher amount of the faster dissolving granulates. The reverse is similarly simple.

The pharmaceutical compositions of the present invention provide a dissolution rate for oxcarabzepine that is similar to a pharmaceutical composition comprising ground oxcarbazepine particles. This is in contrast to the expected dissolution rate for oxcarbazepine particles having a broad particle size distribution, because the pharmaceutical composition of the present invention comprises large/unground oxcarbazepine particles. Furthermore, the dissolution rate of oxcarbazepine from the pharmaceutical composition of the present invention is sufficient for gastro-intestinal absorption of oxcarbazpine in the slightly acidic and neutral pH region. Oxcarbazepine dissolves from the pharmaceutical composition of the present invention at a suitable rate. Preferably, at least 30% of the oxcarbazepine in the pharmaceutical composition dissolves from the composition in a simulated gastro-intestinal environment within 60 minutes. More preferably, at least 30% or more, preferably about 40% or more, of the oxcarbazepine in the pharmaceutical composition dissolves from the composition in such environment within 50 minutes, and most preferably at least 20% of the oxcarbazepine is dissolved from the pharmaceutical composition in such environment within 35 minutes. The preferred dissolution rate for oxcarbazepine from the pharmaceutical composition of the present invention in a simulated gastro-intestinal environment may also be described as; a) no more than about 30% of the total amount of oxcarbazepine is dissolved from the composition after 35 minutes of measurement in a dissolution apparatus; b) from about 30%, or preferably about 40% or more, to about 50% of the total amount of oxcarbazepine is dissolved from the composition after 50 minutes of measurement in a dissolution apparatus; and c) from about 30% to about 50% of the total amount of oxcarbazepine is dissolved from the composition after 60 minutes of measurement in a dissolution apparatus. The gastro-intestinal environment is simulated by using a physiological surfactant in a medium containing an acid such as HCl and a gradual addition of volume and a gradual pH change over time. For example the simulated gastro-intestinal conditions may be as described in Example 14 below.

The pharmaceutical compositions of the present invention provide a bioequivalent formulation to Trileptal®. Pharmaceutical compositions of this embodiment of the present invention that are bioequivalent to Trileptal® comprise oxcarbazepine, a hydrophilic polymer and at least one pharmaceutical excipient, wherein the ratio of hydrophilic polymer to oxcarbazepine is from about 1:3 to about 1:25, preferably from about 1:5 to about 1:22, more preferably from about 1:9 to about 1:20. Furthermore, the disintegration rate of oxcarbazepine from the pharmaceutical composition of the present invention is sufficiently reduced to minimize its influence on bioavailability. The pharmaceutical composition comprising oxcarbazepine of the present invention has a suitable disintegration time as determined by the method described in the USP Pharmacopeia. (USP Pharmacopeia (2007); USP 30, NF 25, vol 1; <701> Disintegration, pp 276-277). Preferably, disintegration time is less than about 30 minutes, more preferably less than about 20 minutes, most preferably less than about 15 minutes.

Oxcarbazepine is used for the treatment of epilepsy in patients suffering from epileptic seizures. The pharmaceutical compositions of the present invention provide an effective delivery system for the administration of oxcarbazepine to patients in need of such treatment. Treatment of patients suffering from epilepsy may comprise administering an effective amount of oxcarbazepine in a pharmaceutical composition of the present invention. Preferably, the pharmaceutical composition comprises a broad particle size distribution of oxcarabazepine, more preferably the broad oxcarbazepine particle size distribution is a multi-modal oxcarbazepine particle size distribution. In addition, oxcarbazepine has been shown to be effective to treat Parkinson's disease. The pharmaceutical composition of the present invention therefore also provides an effective delivery system for the administration of oxcarbazepine to patients suffering from Parkinson's disease and neuropathic pains.

The following examples are presented in order to further illustrate the invention. These examples should not be construed in any manner to limit the invention.

### EXAMPLES

Formulations comprising oxcarbazepine with broad particle size distribution and at least one pharmaceutical excipient were prepared. Some of the examples below demonstrate the perpetration of multi-modal tablet formulations. In order to evaluate the d(0.5) oxcarbazepine particle size distribution of the multi-modal formulation two optional strategies were performed; 1) multi-modal oxcarbazepine mixtures were prepared and their particle size distribution was analyzed using laser diffraction method (Malvern Mastersizer S); or 2) mathematical calculation of d(0.1), d(0.5) and d(0.9) by the calculation of the PSD weighted mean of the large and small oxcarbazepine. The following table describes for example a mathematical calculation of PSD of a multi-modal mixture containing small and large oxcarbazepine particles at a 35:65 ratio, respectively.

TABLE 1
Calculation of particle size distribution containing small and large particles

| Population of small particles | | | Population of large particles | | | Bi-modal | |
|---|---|---|---|---|---|---|---|
| | % of volume | | | % of volume | | | % of volume |
| Particle | with less | | Particle | with less | | Particle | with less |
| Size | than said | | Size | than said | | Size | than said |
| (μm) | particle size | 35% | (μm) | particle size | 65% | (μm) | particle size |
| 0.2 | 7.0 | 2.5 | 0.2 | 0.1 | 0.0 | 0.2 | 2.5 |
| 0.5 | 40.0 | 14.0 | 0.5 | 1.0 | 0.7 | 0.5 | 14.7 |
| 2.0 | 71.7 | 25.1 | 2.0 | 4.1 | 2.7 | 2.0 | 27.8 |
| 4.2 | 83.1 | 29.1 | 4.2 | 8.7 | 5.6 | 4.2 | 34.7 |
| 4.9 | 85.2 | 29.8 | 4.9 | 10.3 | 6.7 | 4.9 | 36.5 |
| 12.2 | 95.4 | 33.4 | 12.2 | 25.1 | 16.3 | 12.2 | 49.7 |
| 14.2 | 96.5 | 33.8 | 14.2 | 28.8 | 18.7 | 14.2 | 52.5 |
| 26.2 | 99.5 | 34.8 | 26.2 | 48.9 | 31.8 | 26.2 | 66.6 |
| 30.5 | 99.8 | 34.9 | 30.5 | 55.3 | 35.9 | 30.5 | 70.9 |
| 48.3 | 100.0 | 35.0 | 48.3 | 75.7 | 49.2 | 48.3 | 84.2 |
| 190.8 | 100.0 | 35.0 | 190.8 | 99.8 | 64.8 | 190.8 | 99.8 |
| 222.3 | 100.0 | 35.0 | 222.3 | 100.0 | 65.0 | 222.3 | 100.0 |

Calculation stages:
1. Data: volume under % of the small and large oxcarbazepine.
2. Each volume under % is multiplied by fraction % (35% or 65% in this example).
3. Sum of 35% product + 65% product

### Example 1

### Preparation of Multi-Modal Oxcarbazepine Formulation with a 10:90 Ratio

In a first step 1 a preparation of a start dispersion from large Oxcarbazepine was prepared. 1200 g of large Oxcarbazepine raw material (RM) [d(0.9)=248.5] was dispersed in a solution of 240 g hypromellose (Pharmacoat 603) in 4800 g of purified water. The Pharmacoat was added to the water and mixed until a clear mixture was obtained. Subsequently, the large Oxcarbazepine was added into the Pharmacoat water mixture and dispersed using a rotor stator (Brogtec) for about 30 min. The large Oxcarbazepine particle size distribution, as measured by the laser diffraction method (Malvern Mastersizer S), was as follows:

| Oxcarbazepine | d(0.1) | d(0.5) | d(0.9) |
|---|---|---|---|
| Large Oxcarbazepine | 25.1 | 86.1 | 308.1 |

In a second step 2 the particle size of the large oxcarbazepine was reduced by a high pressure homogenization process (MFIC microfluidizer M-110Y). This particle size reduction by wet milling was carried out in the presence of a hydrophilic polymer or stabilizer as hypromellose (Hydroxy propyl Methyl Cellulose, for example Pharmacoat®). The particle size distribution of a final Oxcarbazepine dispersion (small Oxcarbazepine), as measured by the laser diffraction method (Malvern Mastersizer S), was as follows:

| Oxcarbazepine | d(0.1) | d(0.5) | d(0.9) |
|---|---|---|---|
| small oxcarbazepine | 0.3 | 0.6 | 2.2 |

In a third step 3 the small Oxcarbazepine final dispersion was used as spray granulation dispersion in fluid-bed-top-spray-granulation process. The granulate formulation contains the following substances:

| Batch # 1 | Amount/dose | |
|---|---|---|
| Oxcarbazepine [as sprayed dispersion (from step 2)] | 600 | mg |
| Pharmacoat [as sprayed dispersion (from step 2)] | 120 | mg |
| Avicel PH 101 | 200 | mg |
| Lactose monohydrate 100 mesh | 140 | mg |
| Starch 1500 NF | 90 | mg |
| Sodium starch glycolate | 50 | mg |

In a fourth step 4 a granulate of large Oxcarbazepine was prepared in a high sheer mixer. The formulation of large oxcarbazepine granulate was as follows:

| | |
|---|---|
| Batch # 2 | Amount/dose |

Part 1

| | | |
|---|---|---|
| large oxcarbazepine [d(0.9) = 248.5] | 600 | mg |
| Avicel PH 101 | 120 | mg |
| Pharmacoat 603 in aqueous granulation solution | 16 | mg |

| | |
|---|---|
| Purified water | q.s. |

Part 2

| | | |
|---|---|---|
| Avicel PH 102 | 14 | mg |
| Crosspovidone | 38 | mg |
| Aerosil 200 | 4 | mg |
| Total weight | 792 | mg |

The particle size distribution of large Oxcarbazepine RM used for the granulate, as measured by laser diffraction method (Malvern mastersizer S), was as follows:

| Oxcarbazepine | d(0.1) | d(0.5) | d(0.9) |
|---|---|---|---|
| Large Oxcarbazepine (2) | 20.9 | 71.5 | 248.5 |

The final fifth step 5 involved the preparation of granulate mixture and tablets. The sprayed granulate of step 3 was mixed together with the granulate of step 4 in a 9:1 ratio, respectively. In preparing a 600 mg dose a mixture of the two granulates was prepared, wherein the sprayed granulate contained 540 mg of active material and the granulate of step 4 contained 60 mg of active material. Consequently the final formulation, including a lubricant as an additional excipient, was as follows.

| Formulation of example 1 | | |
|---|---|---|
| (Batch #3) | Amount/dose | |
| Large Oxcarbazepine granulate | 79 | mg |
| (batch #2) | | |
| Small Oxcarbazepin granulate | 1086 | mg |
| (batch #1) | (after potency correction) | |
| Magnesium stearate | 15 | mg |
| Total weight | 1180 | mg |

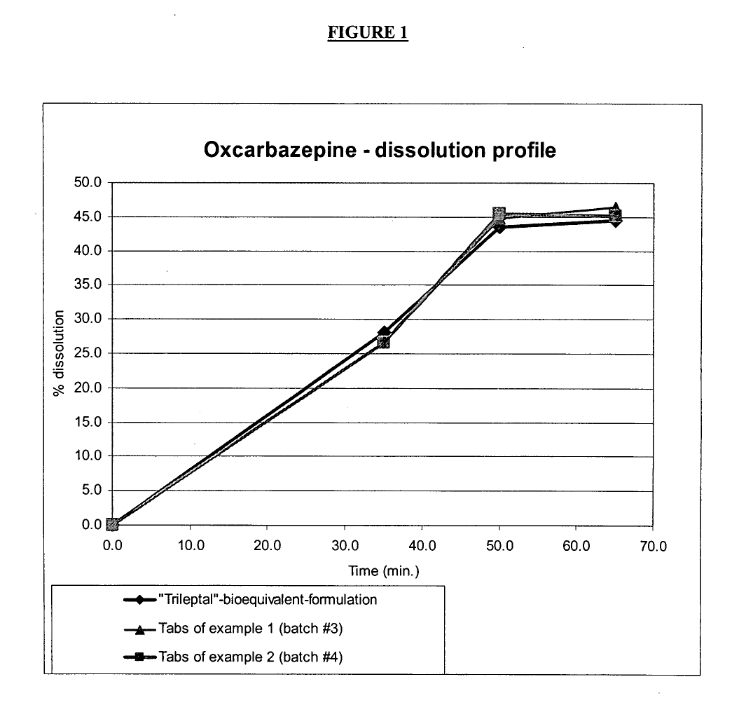

The granulate mixture was subsequently pressed into tablets and a dissolution was performed. The observed dissolution rate was similar to that for the TRILEPTAL® bioequivalent formulation as is shown in FIG. 1.

The particle size of the oxcarbazepine used in this example was estimated to be such that d(0.5) is of the order of 0.6 microns and about 6% of this material would be retained on a 40 micron sieve. The following exemplifies how such estimation is measured. In order to evaluate the d(0.5) of a multi-modal oxcarbazepine formulation with a 10/90 ratio, a mix of two dispersions was performed. The mix contains 90% of small particles characterized by d(0.5) of 0.7 micron and 10% of large particle size characterized by d(0.5) of 68 microns. The d(0.5) of the mix as measured by malvern was 0.7 microns. In order to evaluate the amount of oxcarbazepine retained on a 40 micron screen, sieve analysis of the large oxcarbazepine was performed by alpine. The result of the sieve analysis demonstrates that about 60% of the oxcarbazepine is retained on the 40 micron sieve. Therefore, a mix of 90/10 will retain at least 6% on a 40 micron sieve, assuming that no powder would be retained on the 40 micron mesh screen after sieving of the “small” oxcarbazepine particles.

### Example 2

### Preparation of Multi-Modal Oxcarbazepine Formulation with a 1:5 Ratio

The first four steps were as in example 1. In the final step 5 of preparing a granulate mixture and tablets the sprayed granulate was mixed together with the granulate of step 4 in a 16.6:83.3 ratio, respectively. In preparing a 600 mg dose a mixture of the two granulates was prepared, wherein the sprayed granulate contains 500 mg of active material and the mixed-granulate contains 100 mg of active material. Consequently the final formulation, including a lubricant as an additional excipient, was as follows.

| Formulation of example 2 | | |
|---|---|---|
| (batch #4) | Amount/dose | |
| Large Oxcarbazepine granulate | 132 | mg |
| (batch #2) | | |
| Small Oxcarbazepine granulate | 1005 | mg |
| (batch #1) | (after potency correction) | |
| Magnesium stearate | 15 | mg |
| Total weight | 1152 | mg |

The granulate mixture was subsequently pressed into tablets and a dissolution was performed. The observed dissolution rate was similar to that for the TRILEPTAL® bioequivalent formulation as is shown in FIG. 1. The particle size of the oxcarbazepine used in this example was estimated to be such that d(0.5) is of the order of 0.7 microns and about 10% of this material would be retained on a 40 micron sieve. The following exemplifies how such estimation is measured. In order to evaluate the d(0.5) of a multi-modal oxcarbazepine formulation with a 1:5 ratio, a mix of two dispersions at a close ratio was performed. The mix contains 80% of small particles characterized by d(0.5) of 0.7 micron and 20% of large particle size characterized by d(0.5) of 68 microns. The d(0.5) of the mix as measured by malvern was 0.8 micron. Thus, the d(0.5) of 1:5 ratio is even less than 0.8 micron. In order to evaluate the amount of oxcarbazepine retained on 40 micron sieve, analysis of large oxcarbazepine was performed by alpine. The result of the sieve analysis demonstrates that about 60% of the oxcarbazepine is retained on the sieve. Therefore, a mix of 1:5 will retain about 10% on a 40 micron sieve, assuming that no powder would be retained on 40 micron screen after sieving of the small oxcarbazepine particles.

### Example 3

### Preparation of Multi-Modal Oxcarbazepine Formulation with a 55:45 Ratio using a Combination of a Sprayed Granulate and Mixed Granulate

The first three steps were as in example 1. In step 4 a granulate of Oxcarbazepine [d(0.5)=30.5] was prepared by a wet granulation process. The formulation of large oxcarbazepine granulate was as follows:

| | |
|---|---|
| Batch # 5 | Amount/dose |

Part 1

| | | |
|---|---|---|
| Large oxcarbazepine [d(0.5) = 30.5] | 600 | mg |
| Avicel PH 101 | 120 | mg |
| Pharmacoat 603 in aqueous granulation solution | 16 | mg |

| | |
|---|---|
| Purified water | q.s. |

Part 2

| | | |
|---|---|---|
| Avicel PH 102 | 14 | mg |
| Crosspovidone | 38 | mg |
| Aerosil 200 | 4 | mg |

Part 3

| | | |
|---|---|---|
| Magnesium stearate | 8 | mg |
| Total weight | 800 | mg |

The particle size distribution of large Oxcarbazepine RM used for the granulate, as measured by laser diffraction method (Malvern mastersizer S), was as follows:

| Oxcarbazepine | d(0.1) | d(0.5) | d(0.9) |
|---|---|---|---|
| Large Oxcarbazepine | 4.0 | 30.5 | 62.5 |

The final fifth step 5 involved the preparation of granulate mixture and tablets. The sprayed granulate of step 3 was mixed together with the mixed-granulate of step 4 in a 45:55 ratio, respectively. In preparing a 600 mg dose a mixture of the two granulates was prepared, wherein the sprayed granulate contains 270 mg of active material and the mixed-granulate from step 4 contains 330 mg of active material. Consequently the final formulation, including a lubricant as an additional excipient, was as follows.

| Formulation of example 3 | | |
|---|---|---|
| (batch #6) | Amount/dose | |
| Large Oxcarbazepine granulate | 440 | mg |
| (batch #5) | | |
| Small Oxcarbazepine granulate | 543 | mg |
| (batch #1) | (after potentcy correction) | |
| Magnesium stearate | 10 | mg |
| Total weight | 993 | mg |

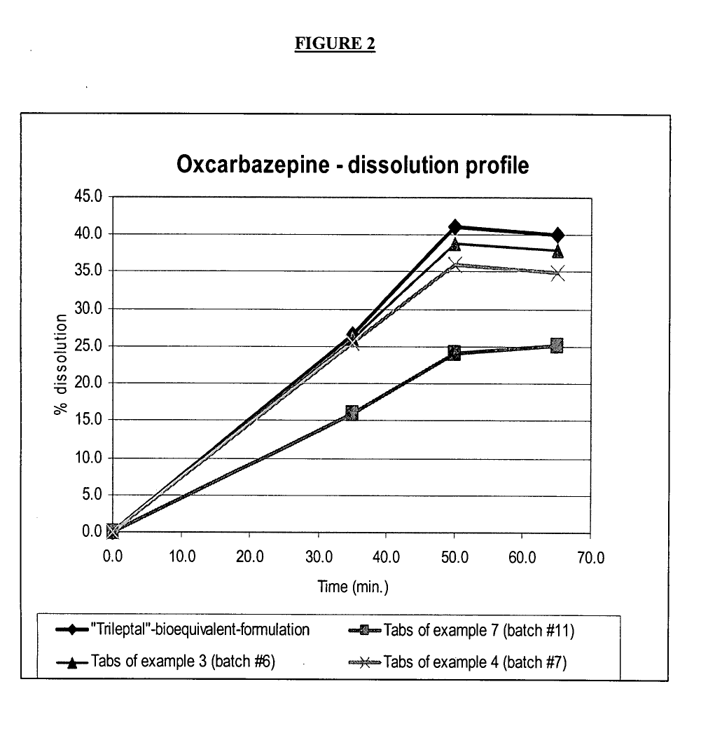

The granulate mixture was subsequently pressed into tablets and a dissolution test was performed. The observed dissolution rate was similar to that for the TRILEPTAL® bioequivalent formulation as is shown in FIG. 2.

### Example 4

### Preparation of Multi-Modal Oxcarbazepine Formulation with a 65:35 Ratio using a Combination of a Sprayed Granulate and Mixed Granulate

The first four steps were as in example 3. In the final step 5 of preparing a granulate mixture and tablets the sprayed granulate was mixed together with the mixed-granulate in a 35:65 ratio, respectively. In preparing a 600 mg dose a mixture of the two granulates was prepared, wherein the sprayed granulate (small) contains 210 mg of active material and the mixed-granulate (large) from step 4 contains 390 mg of active material. Consequently the final formulation, including a lubricant as an additional excipient, was as follows.

| Formulation of example 4 | | |
|---|---|---|
| (batch #7) | Amount/dose | |
| Large Oxcarbazepine granulate | 520 | mg |
| (batch #5) | | |
| Small Oxcarbazepine granulate | 422 | mg * |
| (batch #1) | (after potentcy correction) | |
| Magnesium stearate | 10 | mg |
| Total weight | 952 | mg |

* assayed for oxcarbazepine potency to comprise 210 mg oxcarbazepine

The granulate mixture was subsequently pressed into tablets and a dissolution test was performed. The observed dissolution rate was similar to that for the TRILEPTAL® bioequivalent formulation as is shown in FIG. 2.

In order to estimate the d(0.5) of the 65:35 mixture a mathematical calculation was performed. The d(0.5) of oxcarbazepine contains 65% oxcarbazepine population of d(0.5)=27.6 micron and 35% oxcarbazepine population of d(0.5)=0.8, was calculated. The calculation result indicates that the d(0.5) of that mixture is about 13 microns.

### Example 5

### Preparation of Multi-Modal Oxcarbazepine Formulation with a 65:35 Ratio using a Combination of Two Spray Granulates

The first three steps were as in example 1. In step 4 a dispersion for spray granulation is prepared. 60 g of Large Oxcarbazepine raw material (RM) [d(0.9)=248.5] was dispersed in a solution of 12 g hypromellose (Pharmacoat 603) in 240 g of purified water. The Pharmacoat was added to the water until a clear mixture was obtained. Subsequently, the Large Oxcarbazepine was added into the Pharmacoat water mixture and dispersed using a rotor stator (Brogtec) for about 30 min forming a large oxcarbazepine dispersion. The large Oxcarbazepine particle size distribution following the rotor stator process, as measured by the laser diffraction method (Malvern Mastersizer S), was as follows:

| Oxcarbazepine | d(0.1) | d(0.5) | d(0.9) |
|---|---|---|---|
| Large Oxcarbazepine | 3.3 | 27.6 | 90.4 |

In step 5 the large Oxcarbazepine dispersion was used as spray granulation dispersion in a fluid-bed-top-spray-granulation process. The granulate formulation contains the following substances:

| Batch #8 | Amount/dose | |
|---|---|---|
| Oxcarbazepine [as sprayed dispersion (from step 4)] | 600 | mg |
| Pharmacoat [as sprayed dispersion (from step 4)] | 120 | mg |
| Avicel PH 101 | 250 | mg |
| Lactose monohydrate 100 mesh | 100 | mg |
| Starch 1500 NF | 100 | mg |
| Sodium starch glycolate | 75 | mg |

The final sixth step 6 involved the preparation of granulate mixture and tablets. The sprayed granulate of step 3 was mixed together with the sprayed-granulate of step 5 in a 35:65 ratio, respectively. In preparing a 600 mg dose a mixture of the two granulates was prepared, wherein the sprayed granulate of step 3 contains 210 mg of active material and the sprayed-granulate from step 5 contains 390 mg of active material. Consequently the final formulation, including a lubricant as an additional excipient, was as follows.

| Formulation of example 5 (batch #9) | Amount/dose | |
|---|---|---|
| Large Oxcarbazepine granulate (batch #8) | 836 | mg |
| Small Oxcarbazepine granulate (batch #1) | 422 | mg |
| Magnesium stearate | 10 | mg |
| Total weight | 1268 | mg |

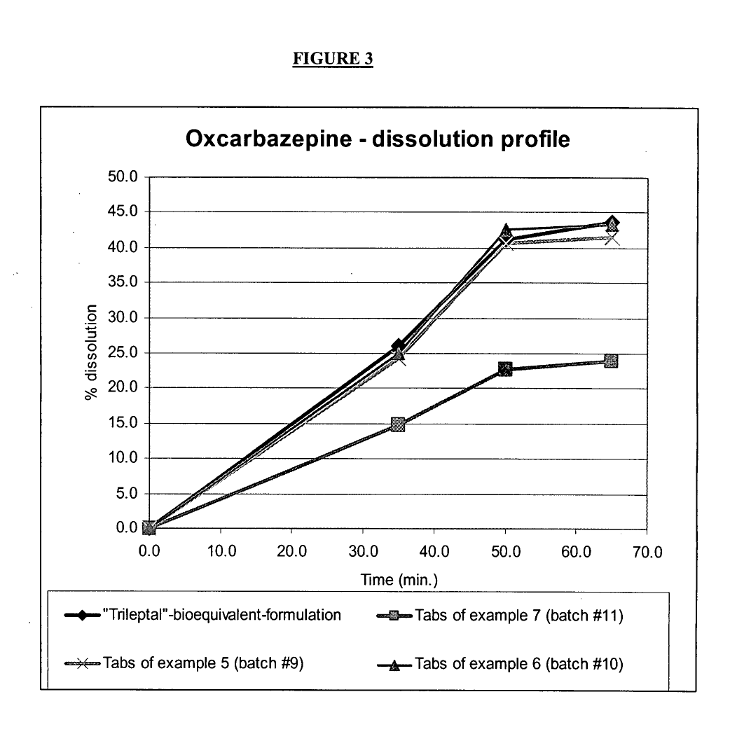

The granulate mixture was subsequently pressed into tablets and a dissolution test was performed. The observed dissolution rate was similar to that for the TRILEPTAL® bioequivalent formulation as is shown in FIG. 3.

In order to evaluate the d(0.5) of the 65:35 mixture, a mathematical calculation was performed. The results of the calculation result indicates that the d(0.5) of the mixture is about 13 microns.

### Example 6

### Preparation of Multi-Modal Oxcarbazepine Formulation with a 55:45 Ratio using a Combination of Two Spray Granulates

The first five steps were as in example 5. In the final step 6 of preparing a granulate mixture and tablets the sprayed-granulate of step 3 was mixed together with the sprayed-granulate of step 5 in a 45:55 ratio, respectively. In preparing a 600 mg dose a mixture of the two granulates was prepared, wherein the sprayed granulate of step 3 contains 270 mg of active material and the sprayed-granulate from step 5 contains 330 mg of active material. Consequently the final formulation, including a lubricant as an additional excipient, was as follows.

| Formulation of example 6 (batch #10) | Amount/dose | |
|---|---|---|
| Large Oxcarbazepine granulate (batch #8) | 707 | mg |
| Small Oxcarbazepine granulate (batch #1) | 543 | mg |
| Magnesium stearate | 10 | mg |
| Total weight | 1260 | mg |

The granulate mixture was subsequently pressed into tablets and a dissolution test was performed. The observed dissolution rate was similar to that for the TRILEPTAL® bioequivalent formulation as is shown in FIG. 3.

### Example 7

### Preparation of Relatively Slow Dissolving Pharmaceutical Composition

A non-bioequivalent formulation with relatively slow dissolving pharmaceutical composition was prepared with oxcarbazepine characterized by d(0.5) of 43 microns. The oxcarbazepine granulate was prepared using high sheer mixer and dried by fluid bed drier. The final formulation, including a lubricant as an additional excipient, was as follows.

| | |
|---|---|
| Formulation of example 7 (batch #11) | Amount/dose |

Part 1

| | | |
|---|---|---|
| Oxcarbazepine | 600 | mg |
| Lactose monohydrate NF 200 mesh | 100 | mg |
| PVP K-30 | 16 | mg |

| | |
|---|---|
| Purified water | q.s. |

Part 2

| | |
|---|---|
| Sodium starch glycolate NF | 10 |
| Avicel PH 102 (microcrystaline cellulose) | 34 |
| Aerosil 200 (colloidal silicone dioxide) | 4 |

Part 3

| | |
|---|---|
| Magnesium stearate | 8 |
| Total Weight | 800 |

The final blend was compressed into tablets and the tablets were coated.

### Example 8

### Preparation of Sprayed Granulated Oxcarbazepine Formulation

In a first step, a preparation of start dispersion from large Oxcarbazepine was prepared. 60 g of Large Oxcarbazepine raw material (RM) [d(0.9)=248.5] was dispersed in a solution of 12 g hypromellose (Pharmacoat 603) in 240 g of purified water. The Pharmacoat was added to the water and mixed until clear mixture was obtained. Subsequently, the Large Oxcarbazepine was added into the Pharmacoat water mixture and dispersed using a rotor stator (Brogtec) for 30 min at 9000 rpm. The Oxcarbazepine particle size distribution following rotor stator process, as measured by the laser diffraction method (Malvern Mastersizer S), was as follows:

| | d(0.1) | d(0.5) | d(0.9) |
|---|---|---|---|
| Large Oxcarbazepine | 5 microns | 27 microns | 76 microns |

In a second step, the large Oxcarbazepine dispersion was used as spray granulation dispersion in fluid-bed-top-spray-granulation process. The granulate formulation contains the following substances:

| Formulation of example 8 (Batch # 12) | Amount/dose | |
|---|---|---|
| Oxcarbazepine [as sprayed dispersion (from step 1)] | 600 | mg |
| Pharmacoat [as sprayed dispersion (from step 1)] | 120 | mg |
| Avicel PH 101 | 250 | mg |
| Lactose monohydrate 100 mesh | 250 | mg |
| Starch 1500 NF | 100 | mg |
| Sodium starch glycolate | 75 | mg |

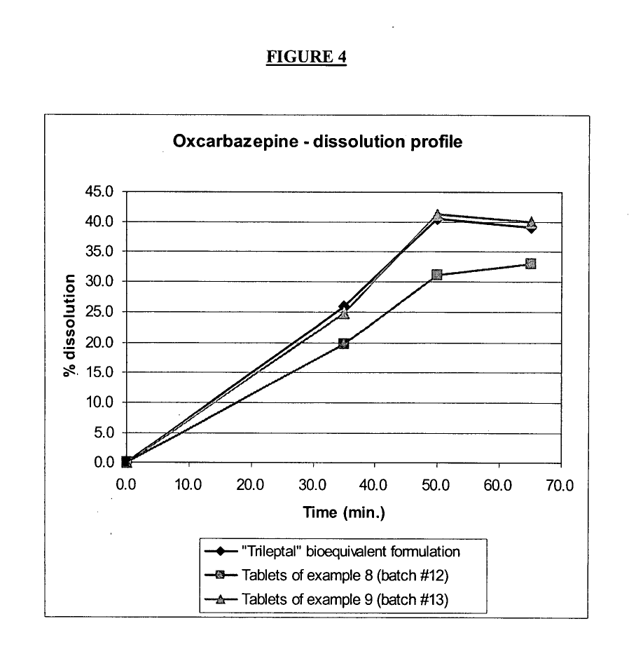

In the third step The granulate mixture was subsequently pressed into tablets and a dissolution test was performed. The observed dissolution rate was almost similar to that for the TRILEPTAL® bioequivalent formulation as is shown in FIG. 4.

The particle size of the oxcarbazepine used in this example was estimated to be such that d(0.5) is of the order of 27 microns and not less than about 29% of this material would be retained on a 40 micron sieve. The following exemplifies how such estimation is reached. In order to evaluate the amount of oxcarbazepine that would be retained on a 40 micron screensieve analysis of large oxcarbazepine was performed by “Fritsch” vibratory sieve shaker for wet sieving. The test was performed with large oxcarbazepine characterized by d(0.5) of 16 microns. The result of the sieve analysis demonstrates that about 29% of the oxcarbazepine would be retained on a 40 micron screen. Therefore, it is obvious that sieving test of large oxcarbazepine used for example 8 and 9 will result by not less than 29% would be retained on 40 micron sieve.

### Example 9

### Preparation of Sprayed Granulated Oxcarbazepine Formulation with Extra-Granular Excipients

The first two steps were as in example 8. In step 3 the oxcarbazepine sprayed granulate was mix together with extragranular excipients as follow:

| Formulation of example 9 (Batch # 13) | Amount/dose | |
|---|---|---|
| Oxcarbazepine sprayed granulate from step 2 [contains | 1659 | mg |
| 600 mg oxcarbazepine (after potency adjustment)] | | |
| Lactose spray dried | 60 | mg |
| Starch 1500 | 25 | mg |
| Crosspovidone | 100 | mg |
| Magnesium stearate | 10 | mg |

In the fourth step The granulate mixture was subsequently pressed into tablets and a dissolution test was performed. The observed dissolution rate was similar to that for the TRILEPTAL® bio equivalent formulation as is shown in FIG. 4.

### Example 10

### Preparation of Sprayed Granulated Oxcarbazepine Formulation

In a first step 1 a preparation of a start dispersion from large Oxcarbazepine was prepared. 30 g of Large Oxcarbazepine raw material (RM) [d(0.9)=248.5] was dispersed in a solution of 6 g hypromellose (Pharmacoat 603) in 120 g of purified water. The Pharmacoat was added to the water and mixed until clear mixture was obtained. Subsequently, the large Oxcarbazepine was added into the Pharmacoat water mixture and dispersed using a rotor stator (Brogtec) for 30 min at 9000 rpm. The large Oxcarbazepine particle size distribution following rotor stator process, as measured by the laser diffraction method (Malvern Mastersizer S), was as follows:

| | d(0.1) | d(0.5) | d(0.9) |
|---|---|---|---|
| Large Oxcarbazepine | 2 microns | 14 microns | 59 microns |

In a second step the large Oxcarbazepine dispersion was used as spray granulation dispersion in fluid-bed-top-spray-granulation process. The granulate formulation contains the following substances:

| Formulation of example 10 (Batch # 14) | Amount/dose | |
|---|---|---|
| Oxcarbazepine [as sprayed dispersion (from step 1)] | 600 | mg |
| Pharmacoat [as sprayed dispersion (from step 1)] | 120 | mg |
| Avicel PH 101 | 110 | mg |
| Lactose monohydrate 100 mesh | 100 | mg |
| Starch 1500 NF | 40 | mg |
| Sodium starch glycolate | 50 | mg |

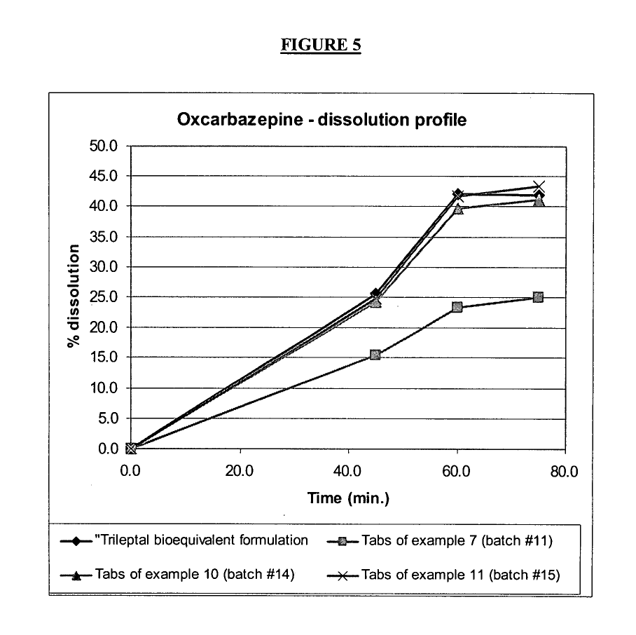

In the third step The granulate mixture was subsequently pressed into tablets and a dissolution test was performed. The observed dissolution rate was similar to that for the TRILEPTAL® bio equivalent formulation as is shown in FIG. 5.

### Example 11

### Preparation of Sprayed Granulated Oxcarbazepine Formulation with Extra-Granular Excipients

The first two steps were as in example 10. In step 3 the oxcarbazepine sprayed granulate was mix together with extragranular excipients as follows:

| Formulation of example 11 (Batch # 15) | Amount/dose | |
|---|---|---|
| Oxcarbazepine sprayed granulate from step 2 [contains | 980 | mg |
| 600 mg oxcarbazepine (after potency adjustment)] | | |
| Lactose spray dried | 120 | mg |
| Starch 1500 | 50 | mg |
| Magnesium stearate | 10 | mg |

In the fourth step The granulate mixture was subsequently pressed into tablets and a dissolution test was performed. The observed dissolution rate was similar to that for the TRILEPTAL® bio equivalent formulation as is shown in FIG. 5.

For formulations prepared as per examples 8, 9, 10 & 11 where a single population of oxcarbazepine is used in a spray granulation process, the particle size of the oxcarbazepine used is preferably such that the d(0.5) value is between about 13 microns and about 30 microns

The following two examples exemplify spray granulated formulations having Oxcarbazepine having d(0.5) less than 2% and greater than 5% that would be retained on a 40 micron sieve.

### Example 12

### Preparation of Multi-Modal Oxcarbazepine Formulation with a 20:80 Ratio wherein a Dispersion of Small Particles of Oxcarbazepine is Sprayed onto Large Oxcarbazepine Particles in a Spray Granulation Process

In a first step 1 a preparation of a start dispersion from large Oxcarbazepine was prepared. 1500 g of Large Oxcarbazepine raw material was dispersed in a solution of 300 g hypromellose (Pharmacoat 603) in 6000 g of purified water. The Pharmacoat was added to the water and mixed until a clear mixture was obtained. Subsequently, the large Oxcarbazepine was added into the Pharmacoat water mixture and dispersed using a rotor stator (Brogtec) for about 30 min. The large Oxcarbazepine particle size distribution, as measured by the laser diffraction method (Malvern Mastersizer S), was as follows:

| | d(0.1) | d(0.5) | d(0.9) |
|---|---|---|---|
| Large Oxcarbazepine | 25 | 68 | 312 |

In a second step 2 the particle size of the large oxcarbazepine was reduced by a high pressure homogenization process (MFIC microfluidizer M-110Y). The particle size distribution of this final Oxcarbazepine (small) dispersion (small Oxcarbazepine), as measured by the laser diffraction method (Malvern Mastersizer S), was as follows:

| | d(0.1) | d(0.5) | d(0.9) |
|---|---|---|---|
| Small Oxcarbazepine | 0.3 | 0.9 | 8.3 |

In a third step 3 the small Oxcarbazepine final dispersion was used as spray granulation dispersion in fluid-bed-top-spray-granulation process and sprayed onto a mix of excipients and large oxcarbazepine. The large Oxcarbazepine particle size distribution, as measured by the laser diffraction method (Malvern Mastersizer S), was as mentioned in stage 1 of the current example.

The granulate formulation contains the following substances:

| Batch # 16 | Amount/dose | |
|---|---|---|
| Small Oxcarbazepine [as sprayed dispersion (from | 480 | mg |
| step 2)] | | |
| Pharmacoat (HPMC) [as sprayed dispersion (from | 96 | mg |
| step 2)] | | |
| Large Oxcarbazepine | 120 | mg |
| Avicel PH 101 | 110 | mg |
| Lactose monohydrate 100 mesh | 100 | mg |
| Starch 1500 NF | 40 | mg |
| Crosspovidone | 50 | mg |

In a fourth step this granulate was mixed together with extra-granular excipients as follows:

| Formulation of example 12 (batch #17) | Amount/dose | |
|---|---|---|
| Oxcarbazepine granulate (batch #16) | 996 | mg |
| Avicel PH 102 | 12 | mg |
| Crosspovidone | 40 | mg |
| Aerosil 200 | 4 | mg |
| Magnesium stearate | 8 | mg |

The granulate mixture was subsequently pressed into tablets and a dissolution test was performed. Additionally, the tablets were coated.

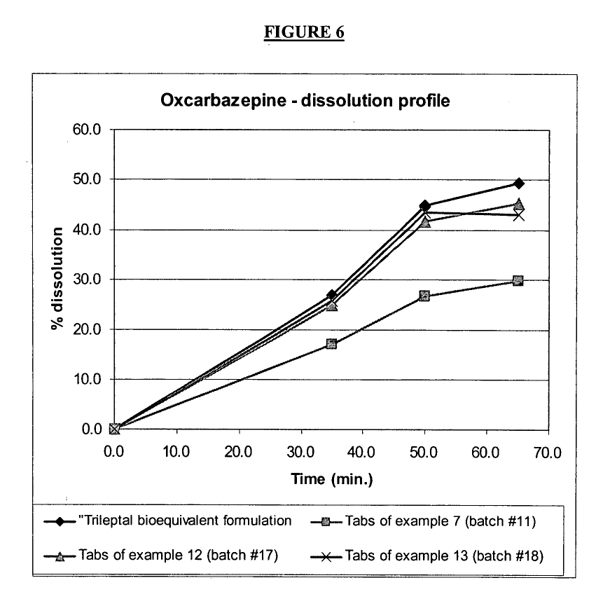

The observed dissolution rate was similar to that for the TRILEPTAL® bioequivalent formulation as is shown in FIG. 6. The particle size of the oxcarbazepine used in this example was estimated to be such that d(0.5) is of the order of 0.8 microns and about 12% of this material would be retained on a 40 micron sieve.

The evaluation of d(0.5) particle size and sieve analysis is demonstrated in example 2.

### Example 13

The first three steps were as in example 12. In step 4 the oxcarbazepine sprayed granulate was mix together with extra-granular excipients as follows:

| Formulation of example 13 (batch #18) | Amount/dose | |
|---|---|---|
| Oxcarbazepine granulate (batch #16) | 996 | mg |
| Lactose spray dried | 60 | mg |
| Starch 1500 | 25 | mg |
| Crosspovidone | 100 | mg |
| Magnesium stearate | 10 | mg |

The granulate mixture was subsequently pressed into tablets and a dissolution was performed. Additionally, the tablets were coated.

The observed dissolution rate was similar to that for the TRILEPTAL® bioequivalent formulation as is shown in FIG. 6. As the granulate is the same as was resultant from example 12 the particle size of the oxcarbazepine used in this example is similary estimated to be such that d(0.5) is of the order of 0.8 microns and about 12% of this material would be retained on a 40 micron sieve.

### Example 14

### Dissolution Test

The tablets described in examples 1-13 were tested in a media containing a physiological surfactant and the procedure included gradual addition of volume and pH changes in order to simulate the gastrointestinal conditions. The dissolution procedure was carried out in an USP Apparatus II, paddle method, at 37° C. and 50 rpm under the following conditions as in Table 2.

TABLE 2
Dissolution procedure conditions.

| Time interval | Media | Volume | |
|---|---|---|---|
| 0-20 min | HCl 0.05N + NaCl 2 g/l | 200 | ml |
| 20-35 min | Lecithin 0.133%, Phosphate buffer, pH 6 | 600 | ml |
| 35-65 min | Lecithin 0.16%, Phosphate buffer, pH 6 | 1000 | ml |

The concentration of the dissolved oxcarbazepine was measured at the 35, 50, and 60 minute time periods. Due to the turbidity of lecithin solutions, the samples are preferably clarified before measurement by UV. To compare the dissolution profile with commercially available pharmaceutical compositions, a pharmaceutical composition which is bioequivalent to TRILEPTAL® was included in the dissolution experiments. Table 4 shows the observed dissolution profile for each of the tablets from examples 1-6, and 8-13, a TRILEPTAL® bioequivalent tablet (“Trileptal® bioequivalent composition A”), and a relatively slow dissolving pharmaceutical composition prepared according to the present invention (example 7).

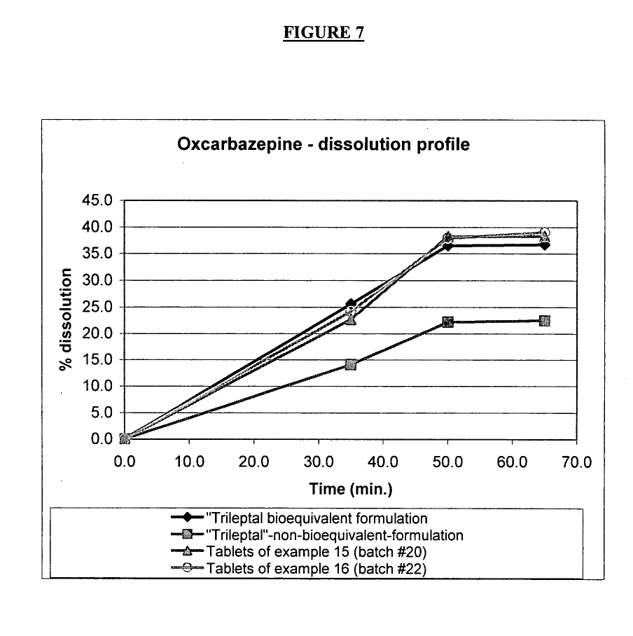

The results shown are obtained in separate experiments. FIG. 1 demonstrates the results of a dissolution test of the TRILEPTAL® bioequivalent composition A versus Examples 1 and 2 under the above described conditions. FIG. 2 demonstrate a dissolution test of the TRILEPTAL® bioequivalent composition A, the relative slow composition (example 7) and Examples 3 and 4. FIG. 3 demonstrates the results of a dissolution test of the TRILEPTAL® bioequivalent composition A, the relative slow composition (example 7), and Examples 5 and 6 under the same dissolution conditions. FIG. 4 demonstrates the results of a dissolution test of the TRILEPTAL® bioequivalent composition A, the relative slow composition (example 7), and Examples 8 and 9 under the same dissolution conditions. FIG. 5 demonstrates the results of a dissolution test of the TRILEPTAL® bioequivalent composition A, the relativly slow composition (example 7), and Examples 10 and 11 under the same dissolution conditions. FIG. 6 demonstrates of a dissolution test of the TRILEPTAL® bioequivalent composition A, the relative slow composition (example 7), and Examples 12 and 13 under the same dissolution conditions. Finally FIG. 7 demonstrates the results of a dissolution test of the TRILEPTAL® bioequivalent composition A, the relative slow composition (example 7), and Examples 15 and 16 under the same dissolution conditions.

Consequently, the values in Table 3 for the TRILEPTAL® bioequivalent composition represent an average of the six experiments and the values in Table 3 for the relatively slow composition represent an average of four experiments. In Table 3 the dissolution profiles of various pharmaceutical compositions are shown in percent oxcarbazepine dissolved from the composition.

TABLE 3
Dissolution profiles of pharmaceutical compositions of oxcarbazepine.

| | Trileptal ® | Relative | | | | | | |
|---|---|---|---|---|---|---|---|---|
| | bioequivalent | slow | | | | | | |
| | composition | composition | | | | | | |
| Time (min) | A | (Example 7) | Example 1 | Example 2 | Example 3 | Example 4 | Example 5 | Example 6 |
| 0.0 | 0.0 | 0.0 | 0.0 | 0.0 | 0.0 | 0.0 | 0.0 | 0.0 |
| 35.0 | 26.9 | 15.4 | 26.7 | 26.7 | 26.1 | 25.5 | 24.4 | 24.9 |
| 50.0 | 42.0 | 23.5 | 44.7 | 45.5 | 38.8 | 35.9 | 40.7 | 42.6 |
| 60.0 | 42.7 | 24.6 | 46.5 | 45.1 | 37.8 | 35.0 | 41.6 | 43.2 |

| Time (min) | Example 8 | Example 9 | Example 10 | Example 11 | Example 12 | Example 13 | Example 15 | Example 16 |
|---|---|---|---|---|---|---|---|---|
| 0.0 | 0.0 | 0.0 | 0.0 | 0.0 | 0.0 | 0.0 | 0.0 | 0.0 |
| 35.0 | 19.7 | 24.9 | 24.3 | 24.8 | 24.8 | 25.7 | 22.7 | 24.2 |
| 50.0 | 31.1 | 41.2 | 39.8 | 41.8 | 41.8 | 43.6 | 38.2 | 37.9 |
| 60.0 | 33.0 | 40.0 | 41.2 | 43.3 | 45.4 | 43.2 | 38.3 | 39.0 |

The bioavaliability of an oral tablet may be influenced by disintegration and dissolution. In order to reduce disintegration time of small particles in oxcarbazepine formulations a reduced amount of hypromellose was used when small oxcarbazepine is sprayed onto large oxcarbazepine particles in a spray granulation process with hypromellose. The following two examples (15 & 16) demonstrate formulations with such reduced amount of hypromellose, which amount of hypromellose is less than in the above examples 1 to 13.

### Example 15

### Preparation of Multi-Modal Oxcarbazepine Formulation with a 20:80 Ratio and 43.2 mg Hypromellose

In a first step 1 a preparation of a start dispersion from large Oxcarbazepine was prepared. 1800 g of Large Oxcarbazepine raw material was dispersed in a solution of 162 g hypromellose (HPMC, Pharmacoat 603) in 3860 g of purified water. The Pharmacoat was added to the water and mixed until a clear mixture was obtained. Subsequently, the large Oxcarbazepine was added into the Pharmacoat water mixture and mixed well. The large Oxcarbazepine particle size distribution, as measured by the laser diffraction method (Malvern Mastersizer S), was as follows:

| | d(0.1) | d(0.5) | d(0.9) |
|---|---|---|---|
| Large Oxcarbazepine | 11 | 79 | 284 |

In a second step 2 the particle size of the large oxcarbazepine was reduced by a high pressure homogenization process (MFIC microfluidizer M-110Y). The particle size distribution of this final Oxcarbazepine (small) dispersion (small Oxcarbazepine), as measured by the laser diffraction method (Malvern Mastersizer S), was as follows:

| | d(0.1) | d(0.5) | d(0.9) |
|---|---|---|---|
| Small Oxcarbazepine | 0.3 | 0.7 | 5.4 |

In a third step 3 the small Oxcarbazepine final dispersion was used as spray granulation dispersion in fluid-bed-top-spray-granulation process and sprayed onto a mix of excipients and large oxcarbazepine. The large Oxcarbazepine particle size distribution, as measured by the laser diffraction method (Malvern Mastersizer S), was as mentioned in stage 1 of the current example.

The granulate formulation contains the following substances:

| Batch #19 | Amount/dose | |
|---|---|---|
| Small Oxcarbazepine [as sprayed dispersion (from | 480 | mg |
| step 2)] | | |
| Pharmacoat 603 (Hypromellose) [as sprayed | 43.2 | mg |
| dispersion (from step 2)] | | |
| Large Oxcarbazepine | 120 | mg |
| Avicel PH 101 | 110 | mg |
| Lactose monohydrate 100 mesh | 100 | mg |
| Starch 1500 NF | 40 | mg |
| Crospovidone | 75 | mg |

In a fourth step this granulate was mixed together with extra-granular excipients as follows:

| Formulation of example 15 (batch #21) | Amount/dose | |
|---|---|---|
| Oxcarbazepine granulate (batch #19) | 968.2 | mg |
| Crospovidone | 150.8 | mg |
| Aerosil 200 | 5.0 | mg |
| Magnesium stearate | 10.0 | mg |

The granulate mixture was subsequently pressed into tablets coated and a dissolution test was performed.

The observed dissolution rate was similar to that for the TRILEPTAL® bioequivalent formulation as is shown in FIG. 7. The d(0.5) of oxcarbazepine used in this example as measured by laser diffraction method (Malvern Mastersizer S) is 0.90 micron and the amount retain on a 40 micron sieve is 13%.

### Example 16

### Preparation of Multi-Modal Oxcarbazepine Formulation with a 20:80 Ratio and 30 mg Hypromellose

In a first step 1 a preparation of a start dispersion from large Oxcarbazepine was prepared. 1800 g of Large Oxcarbazepine raw material was dispersed in a solution of 113 g hypromellose (Pharmacoat 603) in 3800 g of purified water. The Pharmacoat was added to the water and mixed until a clear mixture was obtained. Subsequently, the large Oxcarbazepine was added into the Pharmacoat water mixture and mixed well. The large Oxcarbazepine particle size distribution, as measured by the laser diffraction method (Malvern Mastersizer S), was as follows:

| | d(0.1) | d(0.5) | d(0.9) |
|---|---|---|---|
| Large Oxcarbazepine | 12 | 86 | 320 |

In a second step 2 the particle size of the large oxcarbazepine was reduced by a high pressure homogenization process (MFIC microfluidizer M-110Y). The particle size distribution of this final Oxcarbazepine (small) dispersion (small Oxcarbazepine), as measured by the laser diffraction method (Malvern Mastersizer S), was as follows:

| | d(0.1) | d(0.5) | d(0.9) |
|---|---|---|---|
| Small Oxcarbazepine | 0.3 | 0.6 | 5.0 |

In a third step 3 the small Oxcarbazepine final dispersion was used as spray granulation dispersion in fluid-bed-top-spray-granulation process and sprayed onto a mix of excipients and large oxcarbazepine. The large Oxcarbazepine particle size distribution, as measured by the laser diffraction method (Malvern Mastersizer S), was as mentioned in stage 1 of the current example.

The granulate formulation contains the following substances:

| Batch #22 | Amount/dose | |
|---|---|---|
| Small Oxcarbazepine [as sprayed dispersion (from | 480 | mg |
| step 2)] | | |
| Pharmacoat 603 (Hypromellose) [as sprayed | 30.0 | mg |
| dispersion (from step 2)] | | |
| Large Oxcarbazepine | 120 | mg |
| Avicel PH 101 | 110 | mg |
| Lactose monohydrate 100 mesh | 100 | mg |
| Starch 1500 NF | 40 | mg |
| Crospovidone | 75 | mg |

In a fourth step this granulate was mixed together with extra-granular excipients as follows:

| Formulation of example 16 (batch #23) | Amount/dose | |
|---|---|---|
| Oxcarbazepine granulate (batch #20) | 955.0 | mg |
| Crosspovidone | 150.0 | mg |
| Aerosil 200 | 5.0 | mg |
| Magnesium stearate | 10.0 | mg |

The granulate mixture was subsequently pressed into tablets coated and a dissolution test was performed.

The observed dissolution rate was similar to that for the TRILEPTAL® bioequivalent formulation as is shown in FIG. 7. The d(0.5) of oxcarbazepine used in this example as measured by laser diffraction method (Malvern Mastersizer S) is 0.90 micron and the amount retain on a 40 micron sieve is 13%.

### Example 17

### Study of Single Dose, Comparative Bioavailability of a Composition According to this Invention Compared with Trileptal® 600 mg Marketed Composition

A test of comparative bioavailability on healthy volunteers was carried out.

The following compositions were tested:
1. Compositions according to the invention: coated tablets prepared according to example 15.
2. Marketed Trileptal® 600 mg tablets
   The study was carried out on 15 volunteers receiving a single dose of 600 mg oxcarbazepine, with a minimum 6 days washout period between administrations. The samples for pharmacokinetic analysis were collected in predetermined time intervals. Oxcarbazepine content in plasma was measured for each sample.

The geometric mean of the results and the ratio between them are given in the table below:

| Product | Cmax (ng/mL) | AUC (0-inf) (ng*hr/mL) |
|---|---|---|
| Formulation of example 15 | 1842 | 6818 |
| Formulation of example 16 | 2144 | 6776 |
| Trileptal ® 600 mg tablet | 1888 | 6706 |

| Product | Cmax ratio | AUC (0-inf) ratio |
|---|---|---|
| Ratio: Formulation of | 97.6% | 101.7% |
| example 15/Trileptal ® | | |
| Ratio: Formulation of | 113.6% | 101.0% |
| example 16/Trileptal ® | | |

The presented results_show that Cmax and AUC0-inf ratios of the tested compositions to reference product were found to be within 80% to 125%. Thus, the compositions described in the present invention demonstrated to have comparable bioavailability data to that of the reference Trileptal®.

### Example 18

The disintegration time of the formulations was tested in water using disintegration apparatus (Disintegrator Erweka ZT41).

The results are demonstrated in the following table

| Composition number | Disintegration time (min) |
|---|---|
| Formulation of example 15 | 14:26 min |
| Formulation of example 16 | 7:28 min |

### Example 19

### Study of Single Dose, Comparative Bioavailability of Trileptal® Bioequivelent Composition A Compared with Trileptal® 600 mg Marketed Composition

A test of comparative bioavailability on healthy volunteers was carried out.

The following compositions were tested:

1. Trileptal® bioequivelent composition A.

2. Marketed Trileptal® 600 mg tablets

The study was carried out on 57 volunteers receiving a single dose of 600 mg oxcarbazepine, with a minimum 6 days washout period between administrations. The samples for pharmacokinetic analysis were collected in predetermined time intervals. Oxcarbazepine content in plasma was measured for each sample.

The Cmax and AUC ratios between the test and reference formulations are given in the table below:

| Product | Cmax ratio | AUC (0-inf) ratio |
|---|---|---|
| Ratio: Formulation of | 98% | 102% |
| Trileptal ® bioequivlent | | |
| composition A/Trileptal ® | | |

The presented results_show that Cmax and AUC (0-inf) ratios of the tested composition to reference product were found to be within 80% to 125%. Thus, the composition described has comparable bioavailability data to that of the reference Trileptal®.

### Example 20

### Wet Sieve Method for Determining the Amount of Oxcarbazepine Retained on a 40μ Sieve

1. Objective

To measure the amount of Oxcarbazepine retain on 40 μm sieve.
2. Apparatus

sieving apparatus: Fritsch; Vibratory sieve-shaker “analysette 3”

Sieve: 40 μm
3. Method parameters

Sieving time: 3 min

Amplitude: 2

Interval: 5 sec
4. General description of the method:
   Oxcarbazepine was weighed and poured on sieve. Sieve-shaker was activated according to the parameters described above, and began the water flow. At the end of the sieving process, the sieve (containing the remaining Oxcarbazepine) was dried in oven and the amount of Oxcarbazepine retained on the 40 μm sieve was weighed.

1-6. (canceled)

7. A pharmaceutical composition comprising,
a) oxcarbazepine having a broad particle size distribution, and
b) at least one pharmaceutical excipient,
wherein the broad particle size distribution comprises a multi-modal oxcarbazepine particle size distribution.

8-13. (canceled)

14. The pharmaceutical composition according to claim 7, wherein the multi-modal oxcarbazepine particle size distribution comprises at least one population of large oxcarbazepine particles selected from the group consisting of, a population of oxcarbazepine particles with a d(0.5) value of at least 13 microns, a population of unground particles, particles that do not pass a 40 micron sieve, and mixtures thereof, wherein the total amount of oxcarbazepine in the pharmaceutical composition comprises at least about 20% by weight of a population of large oxcarbazepine particles.

15. The pharmaceutical composition according to claim 14, wherein the total amount of oxcarbazepine in the pharmaceutical composition comprises at least about 40% by weight of a population of large oxcarbazepine particles.

16-21. (canceled)

22. The pharmaceutical composition according to claim 7, wherein the multi-modal oxcarbazepine particle size distribution comprises at least one population of small oxcarbazepine particles having a d(0.5) of less than about 6 microns and wherein the total amount of oxcarbazepine in the pharmaceutical composition comprises at least about 80% of small oxcarbazepine particles.

23-26. (canceled)

27. The pharmaceutical composition according to claim 1, further comprising a hydrophilic polymer and wherein the weight ratio of the hydrophilic polymer to oxcarbazepine is from about 1:3 to about 1:25.

28. The pharmaceutical composition according to claim 27, wherein the hydrophilic polymer is Hydroxypropylmethyl cellulose.

29. The pharmaceutical composition according to claim 27, wherein the weight ratio of the hydrophilic polymer to oxcarbazepine is from about 1:5 to about 1:22.

30. The pharmaceutical composition according to claim 29, wherein the weight ratio of the hydrophilic polymer to oxcarbazepine is from about 1:9 to about 1:20.

31. The pharmaceutical composition according to claim 27, wherein the total amount of hydrophilic polymer in the pharmaceutical composition is not more than 120 mg.

32. The pharmaceutical composition according to claim 31, wherein the total amount of hydrophilic polymer in the pharmaceutical composition is from about 20 mg to about 120 mg.

33. The pharmaceutical composition according to claim 32, wherein the total amount of hydrophilic polymer in the pharmaceutical composition is from about 20 mg to about 50 mg.

34. The pharmaceutical composition according to claim 33, wherein the total amount of hydrophilic polymer in the pharmaceutical composition is from about 30 mg to about 45 mg.

35. The pharmaceutical composition according to claim 27, wherein the amount of hydrophilic polymer in the pharmaceutical composition is from about 1 to about 15% by weight of the composition.

36. The pharmaceutical composition according to claim 35, wherein the amount of hydrophilic polymer in the pharmaceutical composition is from about 2 to about 12% by weight of the composition.

37. The pharmaceutical composition according to claim 36, wherein the amount of hydrophilic polymer in the pharmaceutical composition is from about 2 to about 4% by weight of the composition.

38. The pharmaceutical composition according to claim 27, wherein the multi-modal particle size distribution comprises at least one population of small particles.

39. The pharmaceutical composition according to claim 38, wherein the population of small particles is spray granulated.

40. The pharmaceutical composition according to claim 27, wherein the weight ratio of the hydrophilic polymer to the population of small oxcarbazepine particles is from about 1:3 to about 1:20.

41. The pharmaceutical composition according to claim 40, wherein the weight ratio of the hydrophilic polymer to the population of small oxcarbazepine particles is from about 1:5 to about 1:18.

42. The pharmaceutical composition according to claim 41, wherein the weight ratio of the hydrophilic polymer to the population of small oxcarbazepine particles is from about 1:9 to about 1:17.

43. The pharmaceutical composition according to claim 27, wherein the composition is bioequivalent to Trileptal®.

44-61. (canceled)

62. A pharmaceutical composition comprising,
a) spray granulated oxcarbazepine; and
b) at least one pharmaceutical excipient,
wherein the oxcarbazepine in the composition has at least two populations of different particle sizes and at least one of the populations of oxcarbazepine particles, a population of large oxcarbazepine particles, is spray granulated, the population of large oxcarbazepine particles in an amount of at least about 20% by weight of the total amount of oxcarbazepine being selected from the group consisting of a population of oxcarbazepine particles having d(0.1) of about 21 microns, d(0.5) of about 71 microns and d(0.9) of about 248 microns, a population of oxcarbazepine having been subjected to minimal grinding, a population of unground oxcarbazepine, a population of oxcarbazepine having particles with a d(0.5) value greater than 13 microns, and a population of oxcarbazepine particles that do not pass a 40 micron sieve, and mixtures thereof.

63-65. (canceled)

66. A pharmaceutical composition comprising,
a) spray granulated oxcarbazepine; and
b) at least one pharmaceutical excipient,
wherein the oxcarbazepine in the composition has at least two populations of different particle sizes and at least one of the populations of oxcarbazepine particles, a population of small oxcarbazepine particles, is spray granulated, the population of small oxcarbazepine particles in an amount of at least about 20% by weight of the total amount of oxcarbazepine being a population of oxcarbazepine particles having a d(0.5) of less than about 6 microns.

67. The pharmaceutical composition according to claim 66, wherein the amount of the population of small oxcarbazepine particles is at least about 80% by weight of the total amount of oxcarbazepine.

68-78. (canceled)

79. The pharmaceutical composition according to claim 27, having a dissolution profile in an aqueous buffer simulating the gastrointestinal environment of
a) no more than about 30% of the total amount of oxcarbazepine is dissolved from the composition after 35 minutes of measurement in a dissolution apparatus;
b) from about 30% to about 50% of the total amount of oxcarbazepine is dissolved from the composition after 50 minutes of measurement in a dissolution apparatus; and
c) from about 30% to about 50% of the total amount of oxcarbazepine is dissolved from the composition after 60 minutes of measurement in a dissolution apparatus.

80-86. (canceled)

87. The pharmaceutical composition according to claim 27, having a dissolution profile in media simulating the gastrointestinal environment, wherein at least 30% of the total amount of oxcarbazepine is dissolved from the composition within 60 minutes.

88. The pharmaceutical composition according to claim 87, having a dissolution profile in media simulating the gastrointestinal environment wherein at least 40% of the total amount of oxcarbazepine is dissolved from the composition within 60 minutes.

89. The pharmaceutical composition according to claim 27, having a dissolution profile in media simulating the gastrointestinal environment, wherein at least 30% of the total amount of oxcarbazepine is dissolved from the composition within 50 minutes.

90. The pharmaceutical composition according to claim 89, having a dissolution profile in media simulating the gastrointestinal environment, wherein at least 30% of the total amount of oxcarbazepine is dissolved from the composition within 35 minutes.

91-92. (canceled)

93. The pharmaceutical composition according to claim 27, wherein the disintegration time is less than about 30 minutes.

94. The pharmaceutical composition according to claim 93, wherein the disintegration time is less than about 20 minutes.

95. The pharmaceutical composition according to claim 94, wherein the disintegration time is less than about 15 minutes.

96. A method of treating a patient suffering from epileptic seizures comprising administering a therapeutically effective amount of oxcarbazepine in a pharmaceutical composition of claim 27 to a patient in need thereof.

97. A method of treating a patient suffering from Parkinson's disease comprising administering a therapeutically effective amount of oxcarbazepine in a pharmaceutical composition of claim 27 to a patient in need thereof.

98. (canceled)
